(19) United States
(12) Patent Application Publication
(10) Pub. No.: US 2013/0064855 A1
(43) Pub. Date: Mar. 14, 2013

(54) REPROGRAMMING IMMUNE ENVIRONMENT IN BREAST CANCER VIA DENDRITIC CELLS

(71) Applicants: Anna Karolina Palucka, Dallas, TX (US); Jacques F. Banchereau, Montclair, NJ (US); Florentina Marches, Dallas, TX (US); Chun Yu, Dallas, TX (US); SangKon Oh, Baltimore, MD (US); Te-Chia Wu, Dallas, TX (US)

(72) Inventors: Anna Karolina Palucka, Dallas, TX (US); Jacques F. Banchereau, Montclair, NJ (US); Florentina Marches, Dallas, TX (US); Chun Yu, Dallas, TX (US); SangKon Oh, Baltimore, MD (US); Te-Chia Wu, Dallas, TX (US)

(73) Assignee: BAYLOR RESEARCH INSTITUTE, Dallas, TX (US)

(21) Appl. No.: 13/611,976

(22) Filed: Sep. 12, 2012

Related U.S. Application Data
(60) Provisional application No. 61/533,361, filed on Sep. 12, 2011.

(51) Int. Cl. A61K 31/716; A61K 39/00; A61P 35/00

(52) U.S. Cl. 424/277.1; 514/54

(57) ABSTRACT

Compositions and methods for the treatment of cancer disclosed herein. The method of the present invention comprises administration of compositions comprising β-glucan, a natural ligand for dectin-1, to block OX40L expression on tumor associated mDCs by blocking STAT6 phosphorylation. The β-glucan-treated mDCs secrete higher levels of IL-12p70 and do not expand TNFα and IL-13-producing CD4+ T cells, further resulting in inhibition of Th2 responses. Thus, compositions disclosed herein reprogram the function of mDCs in breast tumor microenvironment and turn tumor promoting Th2-type chronic inflammation into Th1-type acute inflammation that are able to reject tumors. The present invention finds particular uses for the intratumoral administration of the composition thereby directly binding to and directing a Th1-type acute inflammation.

### CROSS-REFERENCE TO RELATED APPLICATIONS

This application claims priority to U.S. Provisional Application Ser. No. 61/533,361, Filed Sep. 12, 2011, the entire contents of which are incorporated herein by reference.

### TECHNICAL FIELD OF THE INVENTION

The present invention relates in general to the field of cancer treatment, and more particularly, to compositions and methods for the treatment of breast cancers by using β-glucans and related compounds to reprogram the breast tumor microenvironment, by a transformation of the tumor promoting (T helper 2) Th2-type cells to (T helper 1) Th1-type cells as well as a specific subset of CD8+ T cells that are able to reject the tumors.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with the treatment of cancer.

Despite declining mortality rates, breast cancer ranks second among cancer related deaths in women. Worldwide, it is estimated that more than one million women are diagnosed with breast cancer every year, and more than 410,000 will die from the disease (1). Immunotherapy could represent an attractive option for treatment-resistant breast cancer (2) (3-6). Indeed, the last decade brought about the demonstration that breast cancer is immunogenic (7, 8). Perhaps the most compelling evidence for effective anti-tumor immunity in breast cancer comes from studies on paraneoplastic diseases (9). Onconeural antigens, which are normally expressed on neurons, the immune privileged sites, are also expressed in some cases of breast cancer (10). In these patients, a strong antigen-specific CD8⁺ T cell response is generated, which provides effective tumor control but also an autoreactive neurologic disease, paraneoplastic cerebellar degeneration (11). Nevertheless, in the majority of cases, the natural immunity to breast cancer is not protective, highlighting the need to develop strategies to boost immune resistance to cancer.

One approach to boosting patient resistance could be vaccination. Indeed, cancer vaccines are in a renaissance era prompted by recent phase III clinical trials showing clinical benefit to the patients. Vaccines act through dendritic cells (DCs) that induce, regulate and maintain T cell immunity. Critical to the design of improved vaccines is the demonstration that DCs are composed of distinct subsets (12-17) which respond differentially to distinct activation signals, (functional plasticity) (18), both contributing to the generation of unique adaptive immune responses. Thus, in the steady state, non-activated (immature) DCs present self-antigens to T cells, which leads to tolerance (19, 20).

Ex vivo cell compositions for providing cancer immunotherapy have also been previously described. For e.g., U.S. Pat. No. 7,402,431 issued to Harnoy (2008) relates to a composition comprising an effective amount of cells, of which a portion are T-cells, whereby said T-cells are contacted with one or more first agents that ligate a cell surface moiety on at least a portion of the T-cells, and whereby the first agents are cross-linked by a second agent attached to a biodegradable support to activate the T-cells, and whereby said activated T-cells and biodegradable supports are suspended in a medium suitable for parenteral infusion in an activated state at a concentration of at least 10⁷ cells per ml of the medium and packaged in a suitable container for administration of said composition to a patient.

Once activated (mature), antigen-loaded DCs are geared towards the launching of antigen-specific immunity (21, 22) leading to the proliferation of T cells and their differentiation into helper and effector cells. The two major subsets are the myeloid DCs (mDCs) and the plasmacytoid DCs (pDCs). pDCs are considered as the front line in anti-viral immunity owing to their capacity to rapidly produce high amounts of type I interferon (23, 24). The best studied human mDC subsets in the tissue are those from skin, where three subsets can be identified. The epidermis hosts only Langerhans Cells (LCs) while the dermis displays two mDC subsets, CD1a⁺ DCs and CD14⁺ DCs, as well as macrophages (17, 25-27). CD14⁺ dermal DCs specialize in generation of humoral immunity with IL-12 being a major cytokine (12) (17), whereas LCs specialize in the priming of high avidity antigen-specific CD8⁺ T cells (17). mDCs can be further polarized by other cells and their products. For example, IL-10 polarized-mDCs generate anergic CD8+ T cells that are unable to lyse tumors (28) as well as CD4+ T cells with regulatory/suppressor function (29). In contrast, thymic stromal lymphopoietin (TSLP)-polarized mDCs are conditioned to expand T cells producing type 2 cytokines (30, 31).

A number of strategies involving the use of dendritic cells (DC) for inducing specific anti-tumor immune responses are being investigated. The use of DC “loaded” with dead cancer cells in vaccine (immunotherapy) approaches has been described in both experimental and clinical settings (see, e.g., Fields et al., Proc Natl Acad Sci USA 95:9482 (1998); Asavaroengchai et al., Proc Natl Acad Sci USA 99:931 (2002); Chang et al., Clin Cancer Res 8:1021 (2002); Geiger et al., Cancer Res 61:8513 (2001)) and others (Eggert et al., Cancer Res 59:3340 (1999); Morse et al., Cancer Res 59:56 (1999); Steinman et al., Nature 449:419 (2007); Steinman, Nature Med 13:1155 (2007)). DC pulsed with tumor-associated antigen(s) in the form of dead tumor cells (denoted TP-DC) can elicit specific T cell proliferation and CTL reactivity, and have shown efficacy in protecting naive mice from tumor challenge and in reducing the growth of tumors in vivo. One such example is provided in U.S. Patent Application Publication No. 2011/0059054 by Mule et al. (2011). The Mule invention discloses a method of preparing a population of enhanced dendritic cells and methods of treating cancer using the enhanced dendritic cells. The method comprises the step of obtaining an initial enriched population of dendritic cells; contacting the dendritic cells with: dead tumor cells; and an antibody or antigen-binding fragment thereof that binds to macrophage receptor with collagenous structure (MARCO), thereby preparing a population of enhanced dendritic cells.

### SUMMARY OF THE INVENTION

The present invention includes compositions and methods for the treatment of cancer. The method of the present invention comprises administration of compositions comprising β-glucan, a natural ligand for dectin-1, to block OX40L expression on tumor associated mDCs by blocking STAT6 phosphorylation. The β-glucan-treated mDCs secrete higher levels of IL-12p70 and do not expand TNFα and IL-13-producing CD4+ T cells, further resulting in inhibition of Th2 responses. The present invention finds particular uses for the intratumoral administration of the composition that binds directly to myeloid dendritic cells, and directing a Th1-type acute inflammation.

A therapeutic composition for a treatment of a tumor of epithelial origin in a human subject is disclosed in one embodiment of the present invention. The composition as described herein comprises one or more active agents that inhibit one or more cytokines that mediate a T helper 2 (Th2) inflammation in the tumor thereby transforming the Th2 inflammation into a tumor rejecting T helper 1 (Th1) type inflammation, wherein the active agent comprises natural or synthetic β-glucans; cyclic β-glucans; β-glucan derivatives; substituted β-glucans; or any combinations thereof optionally solubilized, dispersed, or suspended in a suitable medium in an amount sufficient to treat the tumor.

In one aspect the composition decreases a level of one or more cytokines mediating the Th2 inflammation, wherein the cytokines are selected from the group consisting of IL-4, TNFα, and IL-13 producing CD4+ T cells. In another aspect the composition increases a level or an expression of IL-12p70, CD103+ T cells, or both. In another aspect the active agent binds to a receptor on one or more dendritic cells, wherein the receptor is dectin-1. In a related aspect substituted β-glucans comprise one or more substituents selected from the group consisting of succinyl (Suc) residues; phosphoglycerol residues (P-Gro); phosphoethanolamine (P-Etn) residues; phosphocholine residues (P-Cho); acetyl (Ace) residues; methylmalonyl (MeMal) residues; or any combinations thereof. In yet another aspect the β-glucans may be derived from one or more bacteria selected from the group consisting of Brucella abortus; Brucella melitensis, E. coli; Sinorhizobium melitoti; Bradyrhizobium japonicum; R. sphaeroides; or any combinations thereof.

The composition disclosed hereinabove is used in the treatment of a tumor selected from a breast; prostate; kidney; lung; bladder; colorectal; endometrial; melanoma; thyroid; brain; or pancreatic cancer. In a specific aspect the tumor is breast cancer. In a related aspect the composition is adapted to be administered intratumorally; orally; intravenously; intramuscularly; subcutaneously; intraperitoneally; intradermally; intramucosally; or parenterally. In another aspect the composition reduces a tumor-induced inflammation, inhibits tumor development, or both. In yet another aspect the composition further comprises one or more pharmaceutically acceptable excipients. In another the composition further comprises at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

Another embodiment of the present invention discloses a composition for treating a cancer, ameliorating symptoms of a cancer of epithelial origin in a patient comprising a therapeutically effective amount of one or more active agents that inhibit thymic stromal lymphopoietin (TSLP) mediated OX40L induction, induces IL-12p70 secretion, increases pSTAT4/pSTAT6 ratio, or any combinations thereof, wherein the active agent comprises natural or synthetic β-glucans; cyclic β-glucans; β-glucan derivatives; substituted β-glucans; or any combinations thereof optionally solubilized, dispersed, or suspended in a suitable medium in an amount sufficient to treat or ameliorate the symptoms of the cancer.

In yet another embodiment the present invention provides a therapeutic composition for a treatment, amelioration of symptoms, or both of a tumor of epithelial origin in a human subject comprising one or more active agents that promote myeloid dendritic cells (mDCs) mediated CD8+ T cell expansion and increases expression of CD103, wherein the active agent comprises natural or synthetic β-glucans; cyclic β-glucans; β-glucan derivatives; substituted β-glucans; or any combinations thereof optionally solubilized, dispersed, or suspended in a suitable medium in an amount sufficient to treat or ameliorate the symptoms of the tumor.

The present invention in one embodiment provides a composition for treating, ameliorating symptoms, or both of breast cancer in a human subjects comprising one or more natural or synthetic β-glucans; cyclic β-glucans; β-glucan derivatives; substituted β-glucans; or any combinations thereof optionally solubilized, dispersed, or suspended in a suitable medium in an amount sufficient to treat or ameliorate the symptoms of the breast cancer.

Another embodiment of the present invention relates to a method of treating or ameliorating symptoms of a cancer of epithelial origin in a human subject comprising the steps of: (i) identifying the subject in need of treatment against the cancer and (ii) administering a therapeutically effective amount of a pharmaceutical composition sufficient to treat or ameliorate the symptoms of the cancer in the subject comprising one or more natural or synthetic β-glucans; cyclic β-glucans; β-glucan derivatives; substituted β-glucans; or any combinations thereof optionally solubilized, dispersed, or suspended in a suitable medium.

In yet another embodiment the present invention provides a method of treating an individual who has a tumor, wherein the tumor secretes IL-13 comprising: administering to the individual a therapeutic composition comprising one or more agents that neutralize the IL-13, wherein the one or more agents comprise one or more natural or synthetic β-glucans; cyclic β-glucans; β-glucan derivatives; substituted β-glucans; or any combinations thereof dispersed or solubilized in one or more optional pharmaceutically acceptable excipients.

A method of treating or ameliorating symptoms of breast cancer in a human subject is disclosed in one embodiment of the present invention. The method of the present invention comprises the steps of: identifying the subject in need of treatment against the breast cancer and administering a therapeutically effective amount of a pharmaceutical composition sufficient to treat or ameliorate the symptoms of the breast cancer in the subject comprising one or more natural or synthetic β-glucans; cyclic β-glucans; β-glucan derivatives; substituted β-glucans; or any combinations thereof optionally solubilized, dispersed, or suspended in a suitable medium.

In another embodiment the instant invention provides an ex vivo cellular composition for providing an immunotherapy against one or more tumors of epithelial origin in a human subject comprising one or more β-glucan loaded dendritic cells (DCs) grown or cultured in a medium comprising one or more tumor antigens or factors. In one aspect the DCs comprise myeloid DCs (mDCs). In another aspect the composition inhibits one or more cytokines that mediate a T helper 2 (Th2) inflammation in the tumor thereby transforming the Th2 inflammation into a tumor rejecting T helper 1 (Th1) type inflammation. In yet another aspect the composition decreases a level of one or more cytokines mediating the Th2 inflammation, wherein the cytokines are selected from the group consisting of IL-4, TNFα, and IL-13 producing CD4+ T cells. In a related aspect the composition increases a level or an expression of IL-12p70, CD103+ T cells, or both. In another aspect the β-glucan binds to a receptor on one or more mDCs, wherein the receptor is dectin-1.

A method for providing immunotherapy to a human subject in need of treatment, amelioration of symptoms, or any combinations thereof against breast cancer is also disclosed herein. The method comprises the steps of: (i) identifying the human subject in need of the treatment, amelioration of symptoms, or both against the breast cancer; (ii) isolating one or more dendritic cells (DCs) from the human subject, wherein the DCs are myeloid DCs (mDCs); (iii) contacting the isolated mDCs with a composition comprising one or more natural or synthetic β-glucans; cyclic β-glucans; β-glucan derivatives; substituted β-glucans; or any combinations thereof; and (iv) reintroducing the β-glucan loaded mDCs into the human subject, wherein the loaded mDCs inhibits one or more cytokines that mediate a T helper 2 (Th2) inflammation in the breast cancer thereby transforming the Th2 inflammation into a cancer rejecting T helper 1 (Th1) type inflammation.

In yet another embodiment the instant invention provides a vaccine composition for prevention, treatment, amelioration of symptoms, or any combinations thereof against one or more cancers of epithelial origin comprising: i) an antigen, wherein the antigen comprises a cancer peptide, a tumor associated antigen or any combinations thereof; ii) an adjuvant, wherein the adjuvant comprises an anti-dectin-1 specific antibody or fragment thereof loaded with one or more natural or synthetic β-glucans; cyclic β-glucans; β-glucan derivatives; substituted β-glucans; or any combinations thereof; and iii) a pharmaceutically acceptable carrier, wherein the conjugate and agonist are each comprised in an amount such that, in combination with the other, are effective to produce the immune response in a human or animal subject in need of prevention, treatment, amelioration of symptoms, or any combinations thereof against the one or more cancers of epithelial origin.

A method of preventing, treating, ameliorating symptoms, or any combinations thereof against breast cancer in a human subject is disclosed in the present invention. The method comprises the steps of: i) identifying a human subject in need of prevention, treatment, amelioration of symptoms or any combinations thereof against the breast cancer; and ii) administering therapeutically effective amount of a vaccine composition to the human subject, wherein the composition comprises: (a) an antigen, wherein the antigen comprises a breast cancer peptide, a breast tumor associated antigen or any combinations thereof; (b) an adjuvant, wherein the adjuvant comprises an anti-dectin-1 specific antibody or fragment thereof loaded with one or more natural or synthetic β-glucans; cyclic β-glucans; β-glucan derivatives; substituted β-glucans; or any combinations thereof; and (c) a pharmaceutically acceptable carrier.

In another embodiment the instant invention relates to a composition for use as an adjuvant therapy in a treatment of one or more cancers of epithelial origin comprising one or more natural or synthetic β-glucans; cyclic β-glucans; β-glucan derivatives; substituted β-glucans; or any combinations thereof optionally solubilized, dispersed, or suspended in a suitable medium in an amount sufficient to provide adjuvant therapy following a primary treatment of one or more cancers of epithelial origin. In one aspect the primary treatment comprises surgical intervention; chemotherapy; radiation therapy; immunotherapy; treatment with anti-cancer vaccines; target-specific anti-cancer agents; monoclonal antibodies; or any combinations thereof. In another aspect the cancer is selected from a breast; prostate; kidney; lung; bladder; colorectal; endometrial; melanoma; thyroid; brain; or pancreatic cancer. In yet another aspect wherein the composition reduces a tumor-induced inflammation, inhibits a tumor development, or both.

In yet another embodiment the present invention discloses a method for providing an adjuvant therapy in a human subject following or concurrently with a primary therapy against breast cancer comprising the steps of: identifying the human subject suffering from breast cancer undergoing the primary therapy or who has undergone the primary therapy; and administering a composition comprising one or more natural or synthetic β-glucans; cyclic β-glucans; β-glucan derivatives; substituted β-glucans; or any combinations thereof optionally solubilized, dispersed, or suspended in a suitable medium in an amount sufficient to reduce a tumor-induced inflammation, inhibit a tumor development, or both. In one aspect the primary treatment comprises surgical intervention; chemotherapy; radiation therapy; immunotherapy; treatment with anti-cancer vaccines; target-specific anti-cancer agents; monoclonal antibodies; or any combinations thereof wherein the composition is adapted for intratumoral administration. In another aspect the composition may be used to provide adjuvant therapy in one or more cancers selected from the group consisting of prostate; kidney; lung; bladder; colorectal; endometrial; melanoma; thyroid; brain; or pancreatic cancer.

Yet another embodiment of the present invention is a method of treating or ameliorating symptoms of a cancer of epithelial origin in a human subject comprising the steps of identifying the subject in need of treatment against the cancer; and intratumorally administering a therapeutically effective amount of a pharmaceutical composition in an amount sufficient to treat or ameliorate the symptoms of the cancer in the subject comprising one or more natural or synthetic β-glucans; cyclic β-glucans; β-glucan derivatives; substituted β-glucans; or any combinations thereof optionally solubilized, dispersed, or suspended in a suitable medium. In one aspect, the substituted β-glucans comprise one or more substituents selected from the group consisting of succinyl (Suc) residues; phosphoglycerol residues (P-Gro); phosphoethanolamine (P-Etn) residues; phosphocholine residues (P-Cho); acetyl (Ace) residues; methylmalonyl (MeMal) residues; or any combinations thereof IN another aspect, the β-glucans may be derived from one or more bacteria selected from the group consisting of Brucella abortus; Brucella melitensis, E. coli; Sinorhizobium melitoti; Bradyrhizobium japonicum; R. sphaeroides; or any combinations thereof. In another aspect, the tumor is selected from a breast; prostate; lung; colorectal; or pancreatic cancer.

### BRIEF DESCRIPTION OF THE DRAWINGS

For a more complete understanding of the features and advantages of the present invention, reference is now made to the detailed description of the invention along with the accompanying figures and in which:

FIG. 1 shows the cytokine profile in breast tumor microenvironment;

FIGS. 2A and 2B show the breast cancer immune environment showing inflammatory Th2 response and low CD8 infiltration;

FIGS. 3A to 3E show that breast tumors secret TSLP to induce OX40L expression on mDCs and OX40L+ DCs promote Th2 response;

FIGS. 4A to 4D show that β-glucan modulates mDCs phenotype which includes inhibition of TSLP-mediated OX40L induction, induction of IL-12p70 secretion and pSTAT4/pSTAT6 ratio increase;

FIGS. 5A and 5B show that inflammatory Th2 phenotype in breast tumor condition can be inhibited through β-glucan treatment on mDCs;

FIGS. 6A to 6C show that β-glucan treatment blocks breast tumor growth mediated by immune microenvironment in vivo;

FIGS. 7A and 7B show that β-glucan-treated mDCs expand CD8+ T cells expressing CD103, a marker of intraepithelial T cells;

FIG. 8 is a schematic showing the mechanism by which β-glucan reprograms the immune environment in breast cancer; and

FIG. 9 shows the steps in the in vitro and in vivo assay with β-glucan. In the in vitro assay β-glucan-treated mDCs were activated by breast tumor supernatant to secrete high levels of IL-12p70 and do not expand TNFα and IL-13-producing CD4+ T cells. In the in vivo treatment β-glucan is administered to humanized mice to inhibit breast tumor development.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “myeloid dendritic cell” has its general meaning in the art. The myeloid dendritic cell is an antigen presenting cell existing in vivo, in vitro, ex vivo, or in a host or subject, or which can be derived from a hematopoietic stem cell, a hematopoietic progenitor or a monocyte. The myeloid dendritic cell has a characteristic morphology with thin sheets (lamellipodia) extending in multiple directions away from the dendritic cell body. The main surface markers of human myeloid dendritic cells include CD11b, CD11c, CD33, CD115DCs and BDCA1. The myeloid dendritic cells express constitutively both MHC class I and class II molecules, which present peptide antigens to CD8+ and CD4+ T cells respectively. The myeloid dendritic cell membrane is also rich in molecules that allow adhesion of T cells (e.g. intercellular adhesion molecule 1 or CD54) or that co-stimulate T cell activation such as B7-1 and B7-2 (also known as CD80 and CD86 respectively).

A “receptor” is a naturally occurring molecule or complex of molecules that is generally present on the surface of cells of a target organ, tissue or cell type, e.g., a joint.

The term “cancer” and “cancer cells” refers to any cells that exhibit uncontrolled growth in a tissue or organ of a multicellular organism. The term “breast cancer” is understood to mean any cancer or cancerous lesion associated with breast tissue or breast tissue cells and can include precursors to breast cancer, for example, atypical ductal hyperplasia or non-atypical hyperplasia. The term “tumor” refers to an abnormal benign or malignant mass of tissue that is not inflammatory and possesses no physiological function.

As used herein, the term “β-glucan” (βG) refers to a polysaccharide of D-glucose monomers linked by beta 1,3-glycosidic bonds. Glucans are polysaccharides that only contain glucose as structural components. Beta 1,3-glucans are polysaccharides comprising glucose rings connected at positions 1 and 3 and known as “backbone”. The most active form of beta 1,3-glucans contains 1,6 side-chains in addition to its beta 1,3-glucan backbone and is known as a beta-1,3/1,6 glucan. It is suggested that the configuration of the beta-glucan, including the frequency, location, and length of the side-chains, rather than the backbone of beta-glucans only, determines their immunomodulatory activities. Thus, the term beta-glucan as used with the present invention includes also includes derivatives and variations from one or more sources (e.g., Brucella abortus; Brucella melitensis, E. coli; Sinorhizobium melitoti; Bradyrhizobium japonicum; R. sphaeroides) that are active as taught hereinbelow.

The skilled man in the art can evaluate easily the βG compound having immunoadjuvant properties according to the invention by testing at least one of the biological activities of the invention, such as, for example, cytokine production such as IL-12p and TNF-alpha production and expression of surface marker on Dendritic Cells such as CD80, CD40 and CD86. In second step, induction of CD8+ T cell proliferation and activation can also be tested.

The term “Th2” refers to a subclass of T helper cells that produce cytokines, such as IL-4, IL-5, IL-13, and IL-10, which are associated with an immunoglobulin (humoral) response to an immune challenge.

The term “inflammatory Th2” refers to a subclass of T helper cells that produce IL-4, IL-5, IL-13, and TNFα, and which elicit inflammatory reactions associated with a cellular, i.e. non-immunoglobulin, response to a challenge and which are associated with an immunoglobulin (humoral) response to an immune challenge.

The term “immunotherapy” refers to a treatment regimen based on activation of a pathogen-specific immune response an anti-tumor vaccine as described herein is a form of immunotherapy.

The term “vaccine composition” refers to a composition that can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes or both.

The vaccine could also be used to protect healthy individuals from developing tumors with known antigenic components (“tumor protective vaccine”). In such a case the patient would be treated with known tumor antigens or his own (excised) tumor material targeted in such a fashion to the myeloid dendritic cell of the invention, as to elicit a powerful cytotoxic Th1 immune response against tumor specific antigens. Some vaccines may also be used for desensitization of allergic individuals. Allergic individuals are prone to develop a Th2-overreaction to environmental antigens. The currently available desensitization schemes and treatments aimed at tipping the immune balance to a more Th1-prone immune response to the respective allergen are not fully effective. Therefore, new approaches to induce a more Th1-oriented immunity to the respective allergen(s) are highly desirable. This could be achieved through targeting the respective allergen to the myeloid dendritic cell of the invention that will be capable of eliciting an effective Th1 response to the allergen (“therapeutic desensitization”). A desensitization vaccine could also be applied to individuals who have a predisposition to develop allergic reaction, but have not yet developed allergic symptoms (“preventive desensitization”).

As used herein the term “modify” or “modifies” is meant to include up or down regulation of the function of a gene or gene product, e.g., affecting the transcription, translation, processing, release or modification of a gene or gene product. Examples of modification include, e.g., transcriptional or post-transcriptional silencing, changes to message stability and the like. Examples of post-translational modifications include maturation of the gene product or protein, post-translational modifications (e.g., glycosylation, di-sulfide bonding, myristylation, protease cleavage, association with other proteins, ubiquitination, etc.). The processing, transport and release of the protein may also be modified, e.g., by placing in storage organelles prior to release, by association with other proteins that affect release and the like.

The term “protein” refers to a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless. The term “polypeptide” is a polymer of amino acid residues joined by peptide bonds, whether produced naturally or synthetically. Polypeptides of less than about 10 amino acid residues are commonly referred to as “peptides.”

As used herein the term “antigen” refers to a molecule capable of being specifically bound by an antibody or by a T cell receptor (TCR) if processed and presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “tumor associated antigen” refers to an antigen that is characteristic of a tumor tissue. An example of a tumor associated antigen expressed by a tumor tissue may be the antigen prostatic acid phosphatase (see WO 2004026238) or MART peptide T (melanoma antigen).

The term “antibody” includes, but is not limited to, both naturally occurring and non-naturally occurring antibodies that are isolated and/or purified. Specifically, the term “antibody” includes polyclonal and monoclonal antibodies, and binding fragments thereof that continue to bind to antigen. Furthermore, the term “antibody” includes chimeric antibodies and wholly synthetic antibodies, and fragments thereof. Polyclonal antibodies are derived from the sera of animals immunized with the antigen. Monoclonal antibodies can be prepared using hybridoma technology (Kohler et al., Nature 256:495 (1975); Hammerling et al., in Monoclonal Antibodies and T-Cell Hybridomas, Elsevier, N.Y. pp. 563-681 (1981)). Antibodies also include includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in, e.g., WIPO Publication WO 98/24893, relevant portions incorporated herein by reference.

The term “humanized antibodies” refers to chimeric antibodies that comprise constant regions from human antibodies and hybrid variable regions in which most or all of the framework sequences are from a human variable region and all or most of the CDRs are from a non-human variable region. Humanized antibodies are also referred to as chimeric or veneered antibodies and are produced by recombinant techniques and readily available starting materials. Such techniques are described, for example, in UK Patent Application GB 2,188,638 A, relevant portions incorporated herein by reference.

The term “cytokine” as used herein includes any secreted polypeptide that affects the functions of other cells, and is a molecule that modulates interactions between cells in the immune or inflammatory response. A cytokine includes, but is not limited to monokines and lymphokines regardless of which cells produce them. For instance, a monokine is generally referred to as being produced and secreted by a mononuclear cell, such as a macrophage and/or monocyte but many other cells produce monokines, such as natural killer cells, fibroblasts, ip basophils, neutrophils, endothelial cells, brain astrocytes, bone marrow stromal cells, epideral keratinocytes, and B-lymphocytes. Lymphokines are generally referred to as being produced by lymphocyte cells. Examples of cytokines include, but are not limited to, interleukin-1 (IL-1), tumor necrosis factor-alpha (TNFα) and tumor necrosis factor beta (TNFβ).

The term “pharmaceutically acceptable” includes the carrier, diluent or excipient must be compatible with the other ingredients of the formulation and not deleterious to the recipient thereof.

The terms “administration of” or “administering a” as used herein refers to providing a compound of the invention to the individual in need of treatment in a form that can be introduced into that individual's body in a therapeutically useful form and therapeutically useful amount, including, but not limited to: intratumoral, oral dosage forms, such as tablets, capsules, syrups, suspensions, and the like; injectable dosage forms, such as intradermally, intramucosally (oral, nasal, rectal, etc.), intravenously (IV), intramuscularly (IM), or intraperitonealy (IP), and the like; transdermal dosage forms, including creams, jellies, powders, or patches; buccal dosage forms; inhalation powders, sprays, suspensions, and the like; and rectal suppositories. The composition may be delivered using conventional methods such as one or more pins or a needle using a syringe, or other method of administration such as, ionophoresis, electroporation, or even a dosage gun. The present invention finds particular uses for the intratumoral administration of the composition thereby directly binding to and directing a Th1-type acute inflammation.

The terms “effective amount” or “therapeutically effective amount” refers to the amount of the subject compound that will elicit the biological or medical response of a tissue, system, animal or human that is being sought by the researcher, veterinarian, medical doctor or other clinician.

As used herein, the term “treatment” or “treating” refers to administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, the term “in vivo” refers to being inside the body. The term “in vitro” used as used in the present application is to be understood as indicating an operation carried out in a non-living system.

As used herein, the term “chemotherapeutic” anti-cancer agents are those agents that reduce or eliminate cancer cells and may include, e.g., alkylating/carbamylating agents; platinum derivatives; antimitotic agents; tubulin inhibitors; topoisomerase inhibitors; nucleotide or nucleoside antagonists such as pyrimidine or purine antagonists; and folic acid antagonists.

As used herein, the term “target-specific” anti-cancer agents include those that specifically target cancer cells and include, e.g., taxanes, kinase inhibitors; phspghatase inhibitors; proteasome inhibitors; histone deacetylase inhibitors; heat shock protein inhibitors; vascular targeting agents (VAT); monoclonal antibodies (e.g., Trastuzumab, Rituximab, Alemtuzumab, Tositumomab, Cetuxcimab, Bevacizumab), as well as mutants, fragments and conjugates of monoclonal antibodies (e.g., Gemtuzumab ozogamicin or Ibritumomab tiuxetan); oligonucleotide based therapeutics; Toll-like receptor agonists; protease inhibitors; anti-estrogens hormonal therapeutics; anti-androgens hormonal therapeutics; luteinizing-hormone releasing hormone (LHRH) agents (e.g., Leuprorelin, Goserelin, Triptorelin); aromatase inhibitors; bleomycin; retinoids; DNA methyltransferase inhibitors; alanosine; cytokines; interferons; and death receptor agonists. In one example, the tumor can be a breast cancer and the agent directed to the breast cancer.

Non-limiting examples of anti-cancer agents that may be useful in a combination therapy according to the present invention include, e.g., Actinomycin D, Abarelix, Abciximab, Aclarubicin, Adapalene, Alemtuzumab, Altretamine, Aminoglutethimide, Amiprilose, Amrubicin, Anastrozole, Ancitabine, Artemisinin, Azathioprine, Basiliximab, Bendamustine, Bevacizumab, Bexxar, Bicalutamide, Bleomycin, Bortezomib, Broxuridine, Busulfan, Campath, Capecitabine, Carboplatin, Carboquone, Carmustine, Cetrorelix, Chloram-Bucil, Chlormethine, Cisplatin, Cladribine, Clomifene, Cyclophosphamide, Dacarbazine, Daclizumab, Dactinomycin, Daunorubicin, Decitabine, Deslorelin, Dexrazoxane, Docetaxel, Doxifluridine, Doxorubicin, Droloxifene, Drostanolone, Edelfosine, Eflornithine, Emitefur, Epirubicin, Epitiostanol, Eptaplatin, Erbitux, Erlotinib, Estramustine, Etoposide, Exemestane, Fadrozole, Finasteride, Floxuridine, Flucytosine, Fludarabine, Fluorouracil, Flutamide, Formestane, Foscarnet, Fosfestrol, Fotemustine, Fulvestrant, Gefitinib, Genasense, Gemcitabine, Glivec, Goserelin, Gusperimus, Herceptin, Idarubicin, Idoxuridine, Ifosfamide, Imatinib, Improsulfan, Infliximab, Irinotecan, Ixabepilone, Lanreotide, Letrozole, Leuprorelin, Lobaplatin, Lomustine, Luprolide, Melphalan, Mercaptopurine, Methotrexate, Meturedepa, Miboplatin, Mifepristone, Miltefosine, Mirimostim, Mitoguazone, Mitolactol, Mitomycin, Mitoxantrone, Mizoribine, Motexafin, Mylotarg, Nartograstim, Nebazumab, Nedaplatin, Nilutamide, Nimustine, Octreotide, Ormeloxifene, Oxaliplatin, Paclitaxel, Palivizumab, Patupilone, Pegaspargase, Pegfilgrastim, Pemetrexed, Pentetreotide, Pentostatin, Perfosfamide, Piposulfan, Pirarubicin, Plicamycin, Prednimustine, Procarbazine, Propagermanium, Prospidium Chloride, Raloxifen, Raltitrexed, Ranimustine, Ranpirnase, Rasburicase, Razoxane, Rituximab, Rifampicin, Ritrosulfan, Romurtide, Ruboxistaurin, Sargramostim, Satraplatin, Sirolimus, Sobuzoxane, Sorafenib, Spiromustine, Streptozocin, Sunitinib, Tamoxifen, Tasonermin, Tegafur, Temoporfin, Temozolomide, Teniposide, Testolactone, Thiotepa, Thymalfasin, Tiamiprine, Topotecan, Toremifene, Trail, Trastuzumab, Treosulfan, Triaziquone, Trimetrexate, Triptorelin, Trofosfamide, Uredepa, Valrubicin, Vatalanib, Verteporfin, Vinblastine, Vincristine, Vindesine, Vinorelbine, Vorozole And Zevalin. The person skilled in the art is aware on the base of his/her expert knowledge of the total daily dosage(s) and administration form(s) of the additional therapeutic agent(s) co-administered with the active agents of the present invention and in the methods taught herein. The total daily dosage(s) can vary within a wide range.

When used alone or in combination with an anti-cancer agent, the active agents of the present invention may be provided, separately, sequentially, simultaneously, concurrently or chronologically staggered.

The present invention describes compositions and methods for breast cancer treatment. The present inventors show that β-glucan, a natural ligand for dectin-1, can block OX40L expression on tumor associated mDCs which is due to a block in STATE phosphorylation. The β-glucan-treated mDCs which were activated by breast tumor supernatant secrete higher levels of IL-12p70 and do not expand TNFα and IL-13-producing CD4+ T cells, suggesting the ability to inhibit the Th2 response. Thus, β-glucan can inhibit breast tumor development in humanized mice. The findings presented herein indicate that β-glucan reprogram the function of mDCs in breast tumor microenvironment and turn tumor promoting Th2-type chronic inflammation into Th1-type acute inflammation that are able to reject tumors.

In tumor microenvironment, the crosstalk between infiltrating inflammatory cells and tumor cells creates a cytokine milieu which can promote both oncogenesis and tumor rejection. The present inventors have previously identified in breast cancer tissue the existence of thymic stromal lymphopoietin (TSLP), which promotes tumor development. But how TSLP production is initiated and maintained is not well understood.

The human breast tumor microenvironment displays features of T helper 2 (Th2) inflammation, which promotes tumor development. However, the molecular and cellular mechanisms contributing to Th2 inflammation in breast tumors remain unclear. The inventors have demonstrated that pro-tumor inflammation in breast cancer is driven by breast tumor derived TSLP that induce OX40L expression on mDCs. OX40L+ mDCs generate inflammatory CD4+ T cells producing TNFα and IL-13 but no IL-10. These Th2 cells promote tumor development in vivo in the humanized mouse model of breast cancer which can be inhibited by neutralization of IL-13.

T cells can reject established tumors when adoptively transferred into patients, thereby demonstrating that the immune system can be harnessed for cancer therapy. Active immunotherapy with vaccines has the potential to induce tumor-specific effector and memory T cells that might control tumor outgrowth on the long term. Cancer vaccines are in a renaissance era due to recent phase III clinical trials showing some benefit to the patients. Vaccines act through dendritic cells (DCs) which induce, regulate and maintain T cell immunity. Critical to the design of improved vaccines is the concept of distinct DC subsets and distinct DC activation pathways, all contributing to the generation of unique adaptive immune responses. Rather than the quantity of IFN-g secreting CD8+ T cells, we should aim at generating high quality high sensitivity poly-functional effector CD8+ T cells able to reject tumors and long-lived memory CD8+ T cells able to prevent relapse. Previous studies by the present inventors have actually demonstrated that Langerhans cells are superior, as compared to other DC subsets, in their capacity to prime high affinity melanoma-specific CD8+ T cells able to kill authentic tumor targets.

The recognition of the mechanism of action for beta glucans described herein clarifies that a preferred method for administration of the beta glucans will be intratumoral. Although beta-glucans have been previously suggested as having anti-tumor properties, the focus has heretofore been on oral administration, which will result in a different mode of action of the beta-glucans. In the present invention, it has been unexpectedly shown that the provision of a beta-glucan directly to dendritic cells within a tumor can result in inhibition of the Th2 tumor-promoting inflammatory response, and convert it to a Th1 inflammatory response accompanied by the acquisition of CD8+ T cell response that is capable of rejecting the tumor. Other less direct mechanisms of delivery of beta glucans cannot accomplish the same result, and therefore, the present invention presents a new approach to use of beta glucans for treatment of cancers, particularly epithelial cancers.

The present invention shows that β-glucan, a natural ligand for dectin-1, can block OX40L expression on tumor associated mDCs resulting in secretion of high levels of IL-12p70 and non-expansion of TNFα and IL-13-producing CD4+ T cells, thereby inhibiting the Th2 response. Thus, β-glucan inhibited breast tumor development in humanized mice. Taken these together, the data presented herein suggests that β-glucan reprogram the function of mDCs in breast tumor microenvironment and turn tumor promoting Th2-type chronic inflammation into Th1-type acute inflammation that are able to reject tumors.

DCs can be generated ex-vivo from bone marrow progenitors or blood precursors and loaded with selected antigens for injection to patients. In another approach, DCs can be specifically targeted in vivo with anti-DC antibodies decorated with antigens. This however requires understanding how DCs are affected by the tumor environment.

Another approach to breast cancer immunotherapy could be to block the generation and action of tumor promoting effector T cells secreting type 2 cytokines Indeed, a number of studies in murine models of cancer have demonstrated that type 2 cytokines are involved in tumorigenesis. For example, IL-13 produced by NKT cells induces myeloid cells to make TGF-β which ultimately inhibits CTL functions (Berzofsky and Terabe, 2008). Spontaneous autochthonous breast carcinomas arising in Her-2/neu transgenic mice appear more quickly when the mice are depleted of T cells, evidence for T-cell mediated immunosurveillance slowing tumor growth. This immunosurveillance could be further enhanced by blockade of IL-13, which slowed the appearance of these autologous tumors compared to control antibody-treated mice. A spontaneous mouse breast cancer model recently highlighted the role of Th2 cells, which facilitate the development of lung metastasis through macrophage activation (DeNardo et al. 2009).

The present inventors have previously reported that breast cancer tumor beds are always infiltrated with immature DCs. In contrast, peri-tumoral areas are infiltrated with mature DCs in ˜60% of cases (Bell et al. 1999). The tumor cells polarize DCs into a state that drives the differentiation of naïve CD4⁺ T cells into IL-13-secreting T cells (Aspord et al. 2007). These Type 2 T cells in turn facilitate breast cancer tumor development as shown in xenograft model where it can be partly inhibited by administration of IL-13 antagonists. The present invention show that mDCs respond to breast cancer-derived TSLP by increased expression of OX40L leading to the generation of inflammatory Th2 cells that promote tumor development.

Isolation and culture of myeloid dendritic cells: DCs were purified from buffy coat of blood from healthy donors. Briefly, DCs were enriched from mononuclear cells by negative selection using a mixture of antibodies against lineage markers for CD3, CD14, CD16, CD19, CD56 and glycophorin A (Dynabeads® Human DC Enrichment Kit, Invitrogen). Cells from negative fraction were immuno-labeled with anti-human FITC-labeled lineage cocktail (CD3, CD14, CD16, CD19, CD20 and CD56, BD biosciences Cat. 340546); PE-labeled CD123 (mIgG1, clone 9F5, BD biosciences Cat. 340545), QR-labeled HLA-DR (mIgG2a, clone HK14, Sigma-Aldrich Cat. R8144) and APC-labeled CD11c (mIgG2b, clone S—HCL-3, BD biosciences Cat. 340544). DCs (lin⁻, CD123⁻, HLA-DR⁺, CD11c⁺) were sorted in a FACS Aria cytometer (BD Bioscience). DCs were seeded at 100×10³ cells/well in 200 μl of medium (RPMI supplemented with glutamine 2 mM, penicillin 50 U/ml, streptomycin 50 μg/ml, MEM non-essential amino acids 0.1 mM, HEPES buffer 10 mM, sodium pyruvate 0.1 mM and 10% of human AB serum). DCs were cultured with medium alone or in the presence of 20 ng/ml of TSLP, or different tumor derived products. After 48 hrs DCs were harvested and washed. The stimulated cells were stained for phenotype analysis or co-culture with allogeneic naïve CD4 T cells.

Tumor factors preparation: Tumor factors were obtained from supernatant of HS578T cells cultured in vitro or by sonication from tumor cell lines, human breast tumor tissue or tumors from humanized mice. Briefly, cell lines were culture in medium (RPMI supplemented with glutamine 2 mM, penicillin 50 U/ml, streptomycin 50 μg/ml, MEM non-essential amino acids 0.1 mM, HEPES buffer 10 mM, sodium pyruvate 0.1 mM and 10% of fetal calf serum), and when the cells reached 90% of confluence fresh medium was added and left the cells in culture for additional 48 hrs. For sonication cells or tissues were placed in PBS and were disrupted during 30 seconds at 4° C., with the power output adjusted at 4.5 level of the 60 sonic dismembrator (Fisher Scientific). Cellular debris were removed by centrifugation and the supernatant was collected and stored at −80° C.

Cytokine analysis: Tumor samples from patients diagnosed with breast carcinoma (in situ and invasive duct and/or mucinous carcinoma of the breast, as well as lobular carcinoma) were obtained from the Baylor University Medical Center Tissue Bank. Tumors and draining lymph nodes from humanized mice implanted with breast cancer cell line H578T were also analyzed. Whole-tissue fragments (4×4×4 mm, 0.015-0.030 g, approximately), were placed in culture medium with 50 ng/ml of PMA (Sigma-Aldrich Cat. P8139), and 1 μg/ml of ionomycin (Sigma-Aldrich Cat. 10634) for 18 h. Cytokine production was analyzed in the culture supernatant by Cytokine Multiplex Assay. For intracellular staining, cells were resuspended at a concentration of 10⁶ cells/ml in medium and activated for 5 h with PMA and ionomycin, Brefeldin A (Golgiplug, BD biosciences Cat. 554725) and monensin (Golgistop BD biosciences Cat. 555029) were added for the last 2.5 h.

Tumor bearing humanized mice: CD34+hematopoietic progenitor cells (HPCs) were obtained from apheresis of adult healthy volunteers mobilized with G-CSF and purified as previously described. The CD34− fraction of apheresis was Ficoll purified, and obtained PBMCs were stored frozen and used as a source of autologous T cells. Three million CD34+HPCs were transplanted intravenously into sublethally irradiated (12 cGy/g body weight of 137Cs γ irradiation) NOD/SCID/β2−/− mice (Jackson ImmunoResearch Laboratories). After 4 weeks of engraftment 10 million Hs578T breast cancer cells were harvested from cultures and injected subcutaneously into the flanks of the mice. Mice were reconstituted with 10 million CD4+ T cells and 10 million CD8+ T cells autologous to the grafted CD34+ HPCs. CD4+ and CD8+ T cells were positively selected from thawed PBMCs using magnetic selection according to the manufacturer's instructions (Miltenyi Biotec). The purity was routinely >90%. T cells were transferred at days 3, 6 and 9 post tumor implantation. For experiments with NOD/SCID/β2−/− mice, they were sublethally irradiated the day before tumor implantation. Then mice were reconstituted with 1 million of monocyte derived DCs (MDDCs) and autologous T cells as described above. MDDCs were generated from the adherent fraction of PBMCs by culturing with 100 ng/ml GM-CSF (Berlex) and 10 ng/ml IL-4 (R&D Systems). Tumor size was monitored every 2-3 d. Tumor volume (ellipsoid) was calculated as follows: [(short diameter)²×long diameter]/2.

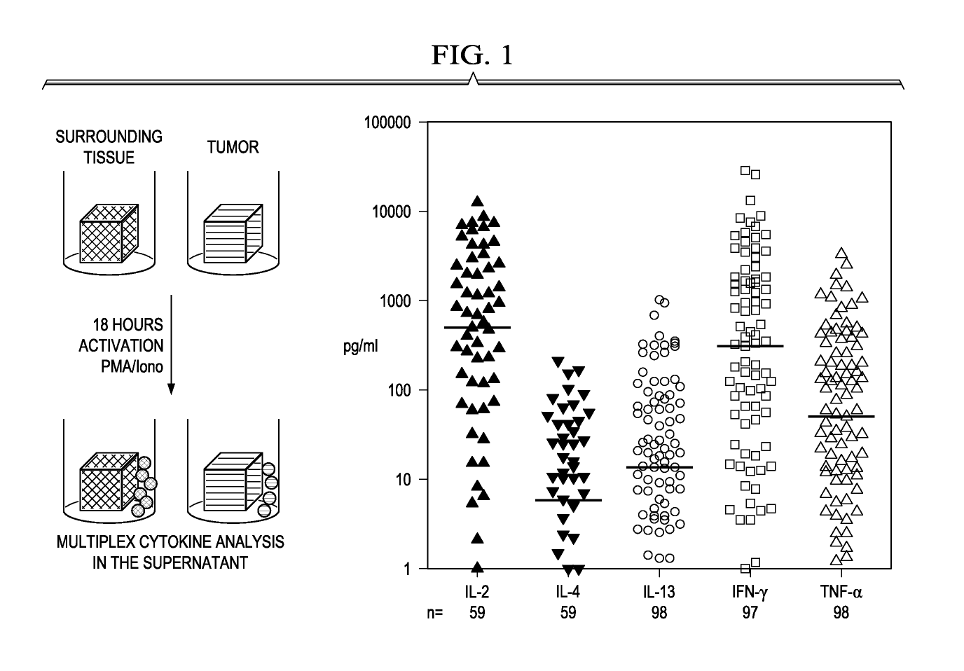

Inflammatory Th2 cells in primary breast cancer tumors: A previous study by the present inventors using a pilot cohort of 19 samples of primary breast cancer tumors revealed the secretion, upon activation with PMA/ionomycin, of both type 1 (IFN-γ) and type 2 (IL-4 and IL-13) cytokines (Aspord et al. 2007). The current study analyzes a total of 99 consecutive samples. Supernatants of activated tumor fragments display high levels of IFN, IL-2, IL-4, IL-13 and TNF (FIG. 1).

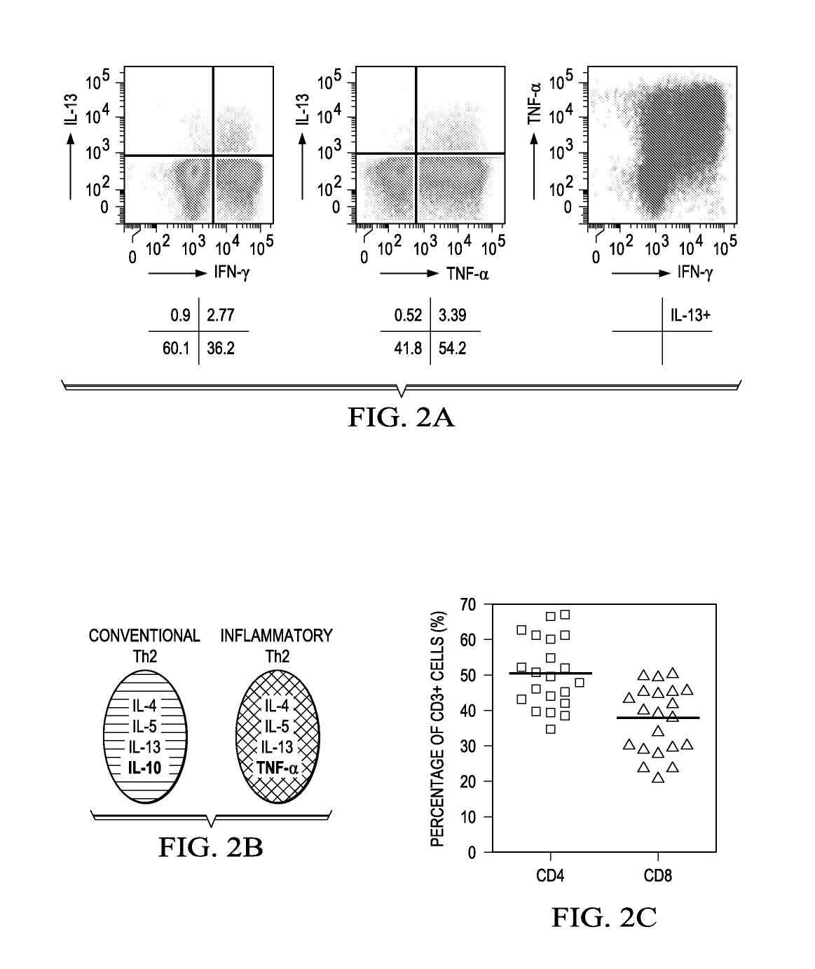

To identify the cells producing these cytokines, single-cell suspensions were prepared from tumors, activated for 5 hrs with PMA/ionomycin, stained with antibodies against T cells and cytokines and analyzed by flow cytometry. Gated viable CD4⁺CD3⁺ T cells expressed IL-13 (3.67%), most of them co-expressing IFN-γ and TNF-a (FIGS. 2 A, 2B, and 2C). A small fraction of IL-13⁺CD4⁺ T cells co-expressed IL-4 but none expressed IL-10 (FIG. 2A). Such T cells have been referred to as inflammatory Th2 cells that are involved in allergic inflammatory diseases (Liu et al. 2007).

The inventors have previously shown that DCs infiltrating breast cancer tumors express OX40 ligand. Soluble OX40L could actually be detected by ELISA in supernatants from sonicated breast cancer tumor fragments (data not shown). Immunofluorescence staining of frozen tissue sections of primary breast cancer tumors showed the expression, in 57 out of 60 analyzed tumors, of OX40L by a majority of HLA-DR^{high} cells (data not shown). These OX40L⁺ cells are located in peri-tumoral areas. Flow cytometry analysis of single cell suspensions further confirmed the expression of OX40L by a fraction of HLA-DR^{high} CD11c^{high} mDCs (data not shown). Paired analysis demonstrated that the tumor beds express higher percentages of OX40L⁺ mDCs than the surrounding tissue (p=0.0156, n=7 paired samples, mean±SE for surrounding tissue=1.5%±0.8, n=7; and for breast cancer tumors 11%±1.67, n=12, respectively). Thus, breast cancer tumors are infiltrated with OX40L⁺ mDCs.

Breast cancer tumors produce soluble factors that induce OX40L on DCs. Breast cancer tumors express and secrete TSLP. OX40L can be induced on mDCs by TSLP, an IL-7 like cytokine produced by epithelial cells (Liu et al. 2007; Ziegler and Artis, 2010). The supernatants of the Hs578T breast cancer cell line contained low levels of TSLP, which could be substantially increased upon activation with PMA/Ionomycin (data not shown). Supernatants of some primary breast cancer tumors activated with PMA/Ionomycin displayed up to 300 pg/ml TSLP (data not shown). The expression of TSLP by cancer cells was further analyzed using an anti-TSLP antibody and immunofluorescence of frozen breast cancer tumors generated in the xenograft model (Aspord et al. 2007). There, subcutaneous MDA-MB-231 tumors transplanted in mice expressed TSLP (data not shown). The specificity of the staining is demonstrated by pre-treatment of the antibody with recombinant TSLP.

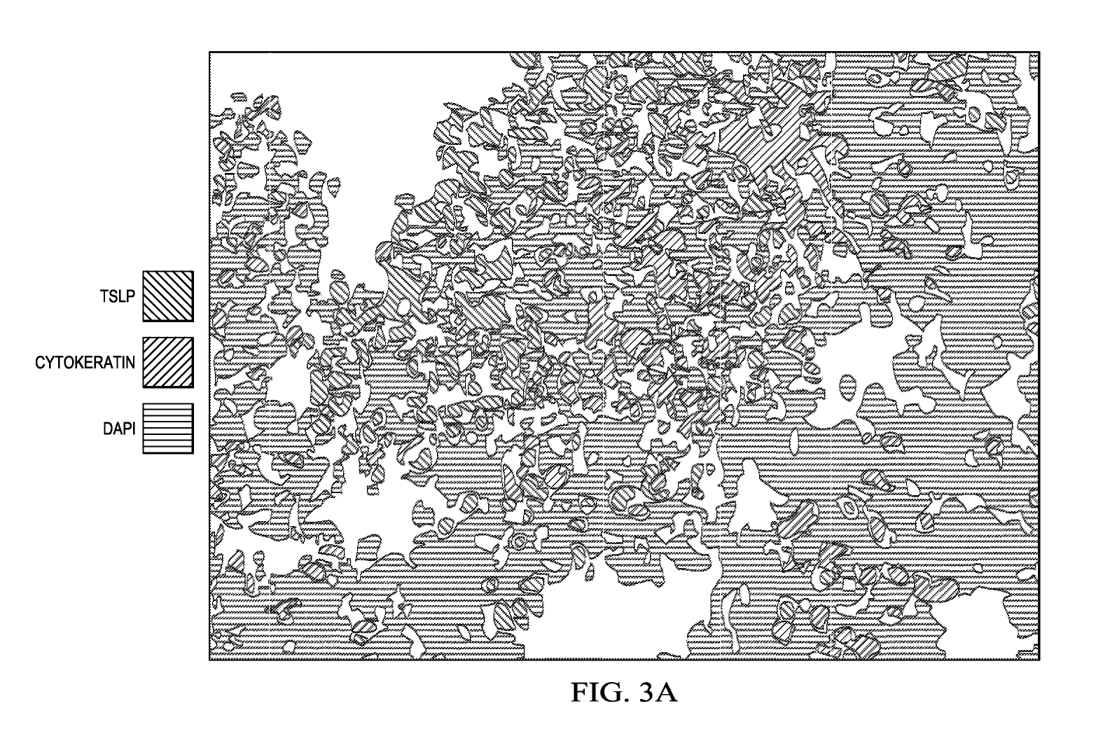

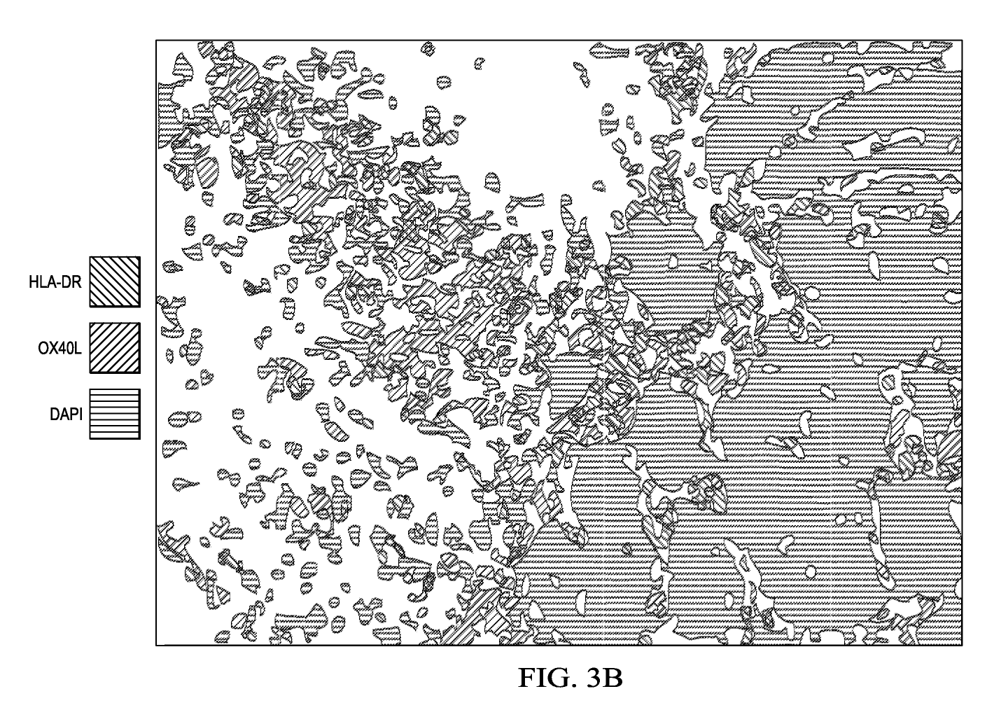

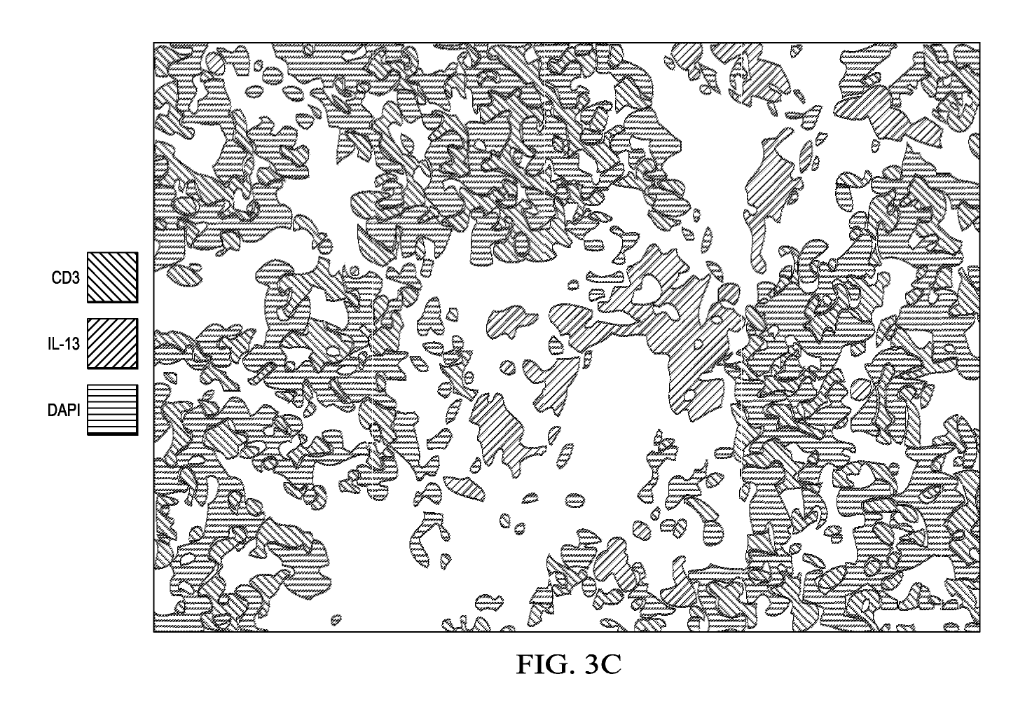

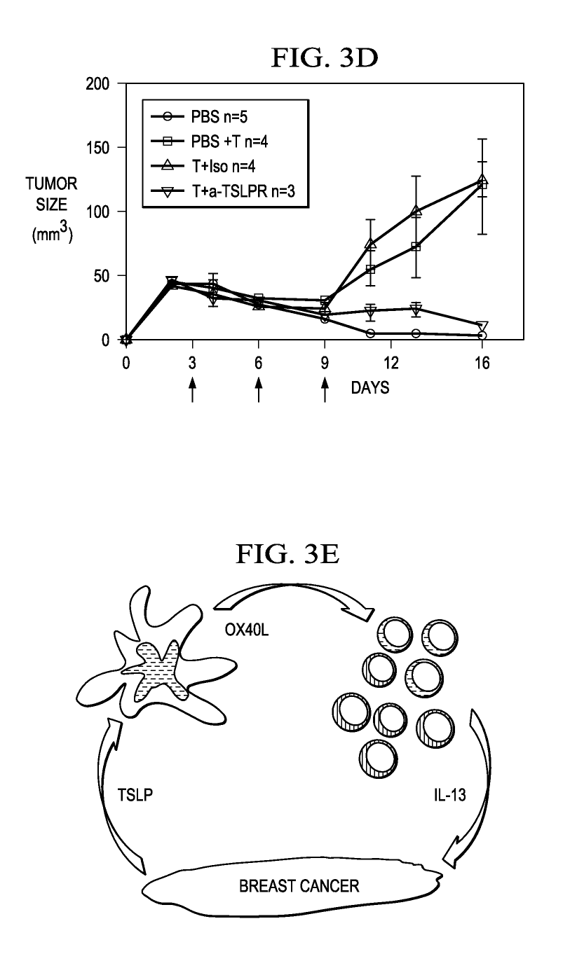

The present inventors have previously shown that TSLP is expressed in 35 out of 38 analyzed primary breast cancer tumors obtained from patients regardless of grade, histology or stage of analyzed tumors. FIGS. 3A, 3B, and 3C illustrate the pattern of TSLP staining and co-expression with cytokeratin 19 positive cells. It demonstrates that TSLP is expressed in the cytoplasm and the nucleus of breast cancer cells that display IL-13 on their surface. Importantly, TSLP is also expressed in lung and kidney metastasis of MDA-MB-231 tumors in humanized mice and in breast cancer tumor metastasis from patients. Thus, similarly to normal skin or lung epithelium, breast cancer cells have the capacity to express, produce and secrete TSLP.

As previously described the present inventors in FIG. 3 E propose a vicious circle of smoldering type 2 inflammation that perpetuates breast cancer and which is maintained by TSLP. There, breast cancer attracts DCs possibly through macrophage inflammatory protein 3 alpha (MIP3-α) (Aspord et al. 2007; Bell et al. 1999). Tumor infiltrating DCs are then exposed to TSLP secreted by breast cancer cells, which triggers their maturation and OX40L expression. This might explain the aseptic mDC maturation as found in breast cancer (Aspord et al. 2007; Bell et al. 1999). OX40L+ mDCs induce CD4+ T cells to secrete IL-13, as well as TNF-α. These inflammatory CD4⁺ T cells contribute to tumor development in an IL13-dependent pathway. Thus far, TSLP represents the only factor that activates mDCs without inducing them to produce Th1-polarizing cytokines (Liu et al. 2007). Under normal physiological conditions, TSLP appears to play a critical role in CD4⁺ T cell homeostasis in the peripheral mucosa-associated lymphoid tissues and in the positive selection and/or expansion of Tregs in the thymus (Watanabe et al. 2005a; Watanabe et al. 2005b). In inflammatory conditions, such as atopic dermatitis and asthma, epithelial cells markedly increase TSLP expression (Liu et al. 2007). The TSLP-activated DCs migrate to the draining lymph nodes, prime CD4⁺ T cells via OX40L to differentiate into inflammatory Th2 effector and memory cells and therefore initiate the adaptive phase of allergic immune responses. Interestingly, in breast cancer OX40L⁺ mDCs are present in the tumor. It remains to be determined whether this reflects their inability to migrate from the tumor to draining lymph nodes. It also remains to be determined whether these DCs are able to prime Th2 immunity in situ in tertiary lymphoid structures or whether their main role is to maintain the activation and survival of Th2 cells at the tumor site. Their ability to maintain Th2 cell phenotype and effector function is supported by our earlier studies showing that T cells isolated from experimental breast tumors and transferred to naïve tumor bearing humanized mice can promote tumor development even at low numbers and upon single injection (Aspord et al. 2007).

The invention will be further illustrated by the following figures and examples. However, these examples and figures should not be interpreted in any way as limiting the scope of the present invention.

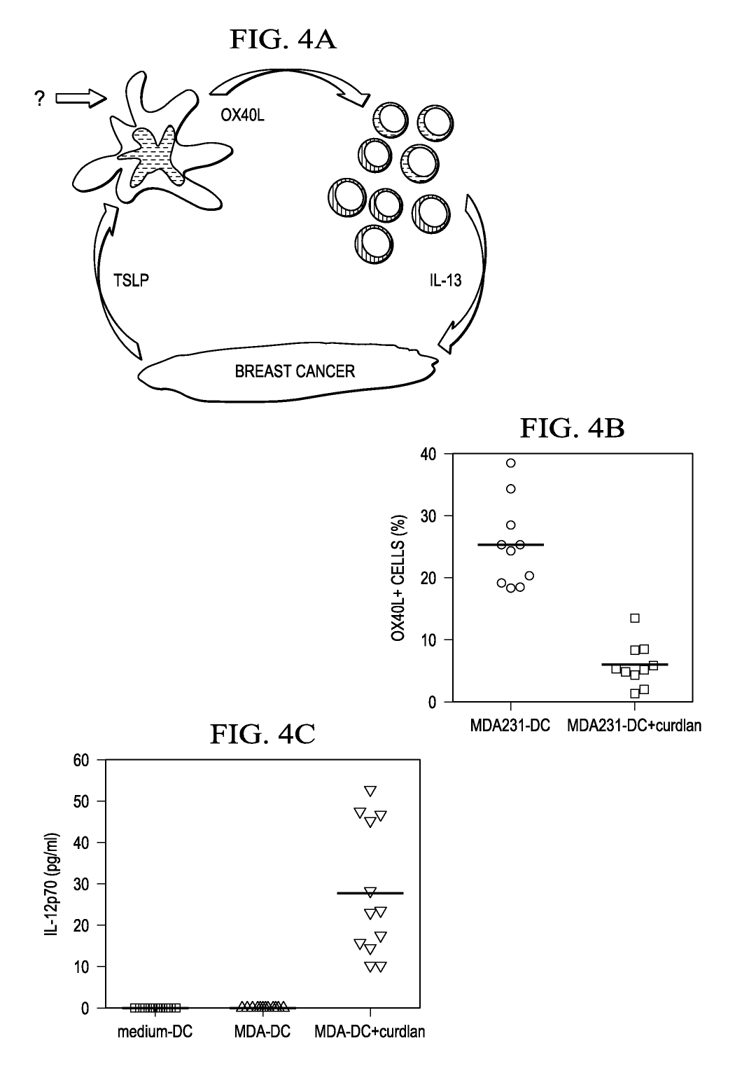

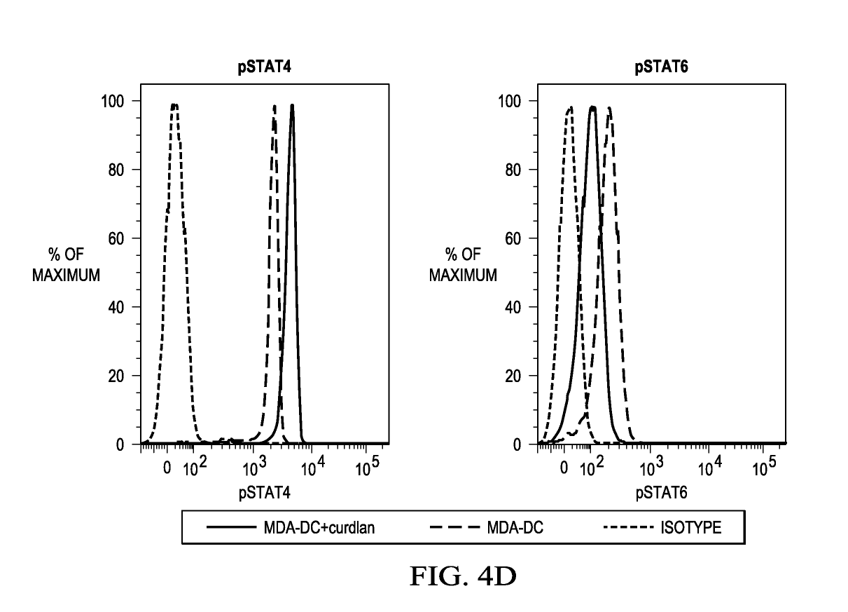

The present inventors believe that DC activators can modulate the functions of DCs and reprogram the immune microenvironment (FIG. 4 A). FIGS. 4B, 4C and 4D show the results when DCs were purified from buffy coat of blood from healthy donors. DCs were cultured with medium alone or in the presence of tumor derived products (FIG. 4B). The curdlan treatment is 3 min at room temperature prior to adding tumor supernatant. After 48 hrs, DCs were harvested and washed. The stimulated cells were staining for phenotype (OX40L, pSTAT4 and pSTAT6). The supernatant of DC culture was used for cytokine (IL-12p70) examination (FIGS. 4C and 4D).

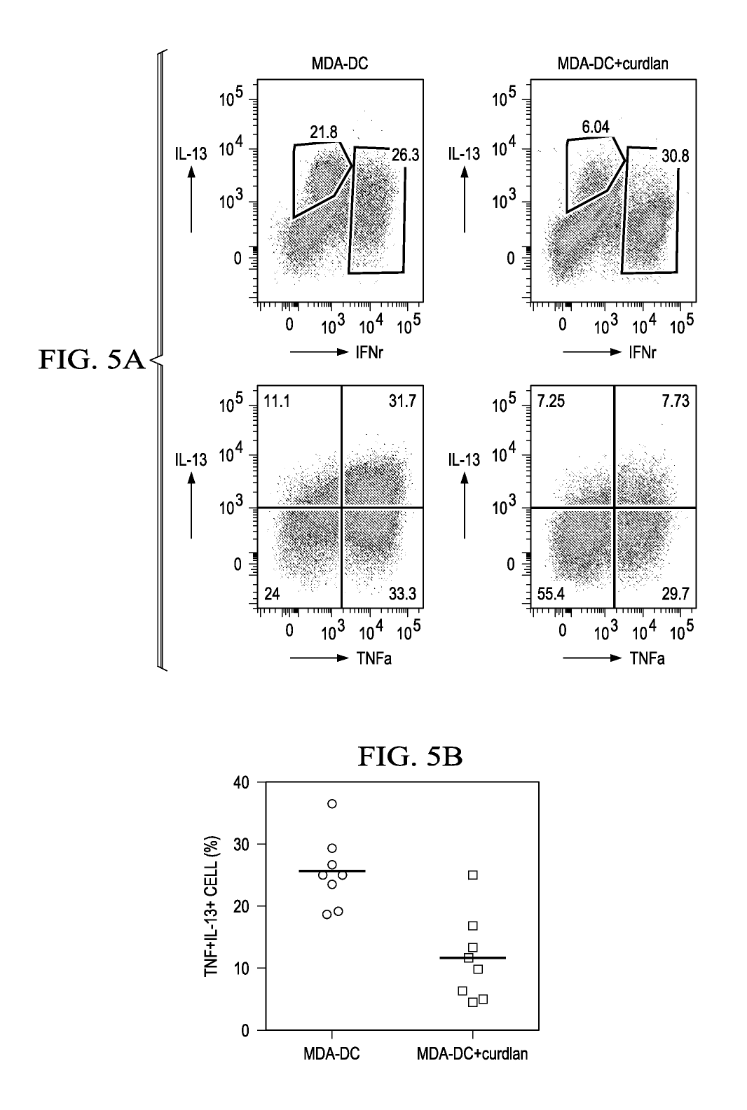

As seen in FIG. 5 A sorted mDCs (HLA-DR+/CD11c+/CD123−/Lin−) were pre-treated with 100 ug/ml curdlan and activated by supernatant of a breast cancer cell line (MDA-MB231) for 48 hrs and then co-cultured with allogenic naive CD4⁺ T cells. After one week, cells were collected and re-stimulated for intracellular cytokines analysis. The analysis of different studies showing the effect of curdlan treatment in the induction of TNFa+IL-13+ secreting cells is shown in FIG. 5 B.

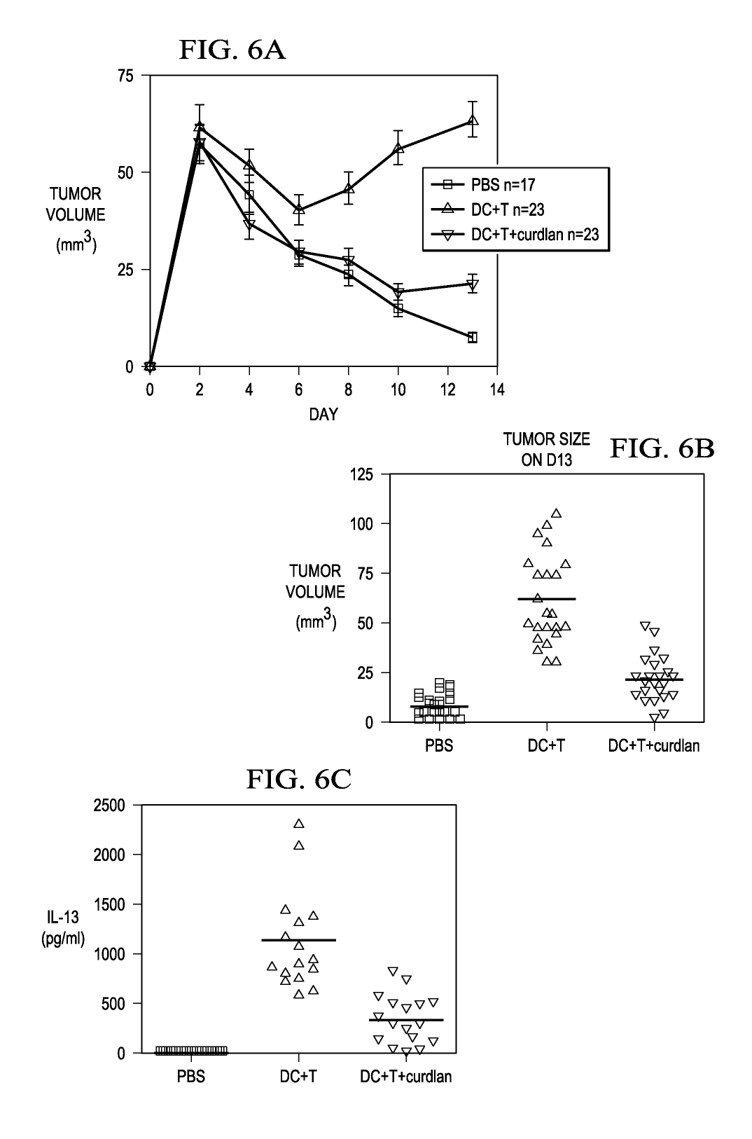

FIGS. 6A and 6B show NOD/SCID/β2−/− mice sub-lethally irradiated the day before tumor implantation. Then mice were reconstituted with 1 million of monocyte derived DCs (MDDCs) and autologous T cells. Curdlan (100 ug/ml) was co-injected with DCs and T cells at days 3, 6 and 9 post tumor implantation. Tumor size was monitored every 2-3 d. Tumor volume (ellipsoid) was calculated as follows: [(short diameter)²×long diameter]/2. In FIG. 6C tumors were harvested on day 14 and stimulated with PMA/Ionomycin for 18 hrs. Supernatant was harvested and cytokines were detected by Luminex.

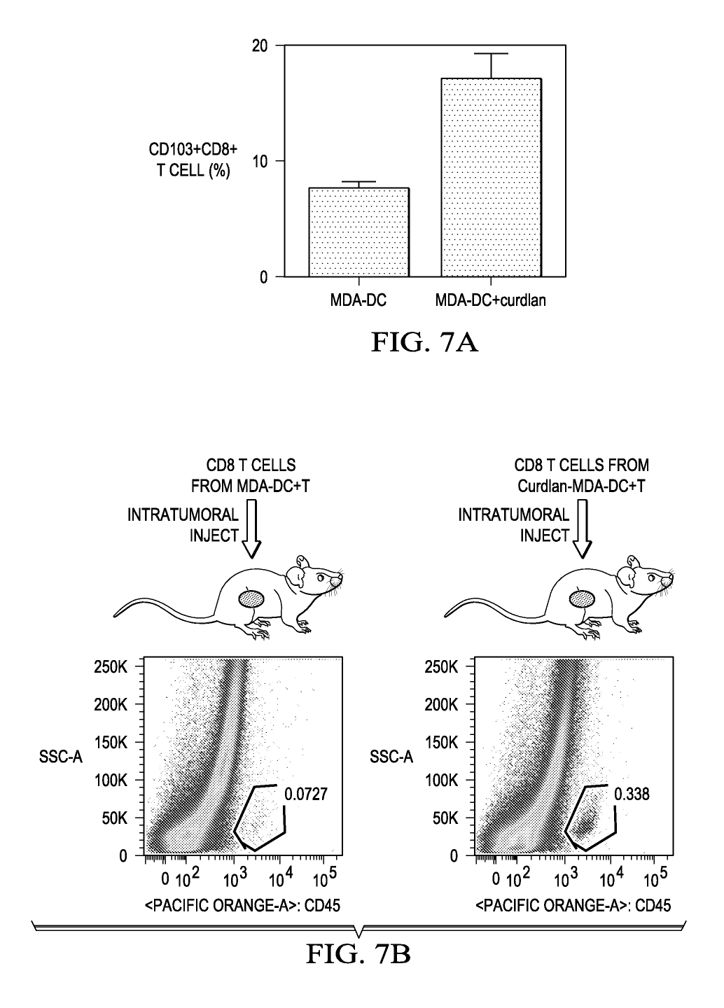

Sorted mDCs (HLA-DR+/CD11c+/CD123−/Lin−) were pre-treated with 100 ug/ml curdlan and activated by supernatant of a breast cancer cell line (MDA-MB231) for 48 hrs and then co-culture with allogenic naive total T cells (FIG. 7A). After one week, cells were collected and stained with intraepithelial marker, CD 103, together with granzymeA and granzymeB staining CD8+ T cells were sorted from in vitro culture and intratumoral inject into NOD-SCID mice. After 3 days, tumors were harvested and digested with collagenase (FIG. 7B).

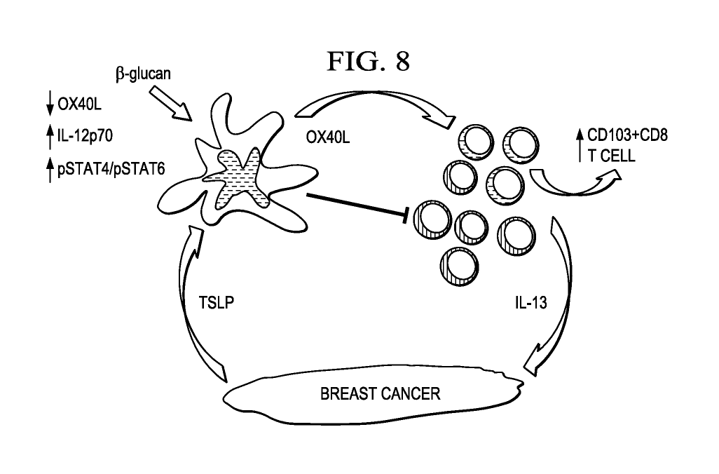

FIG. 8 is a schematic showing the mechanism by which β-glucan reprograms the immune environment in breast cancer.

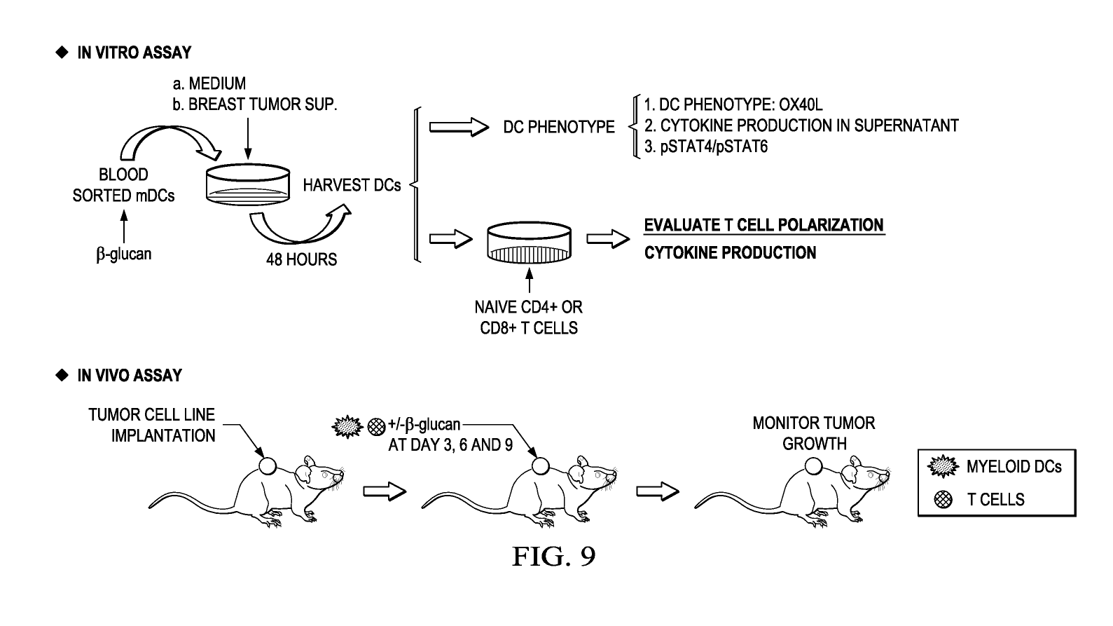

FIG. 9 is a schematic showing the steps in the in vitro and in vivo assay with β-glucan. In the in vitro assay β-glucan-treated mDCs were activated by breast tumor supernatant to secrete high levels of IL-12p70 and do not expand TNFα and IL-13-producing CD4+ T cells. In the in vivo treatment β-glucan is administered to humanized mice to inhibit breast tumor development

A further object of the invention relates to a vaccine composition, comprising the beta-glucan compound according to the invention as an immunoadjuvant optionally with one or more pharmaceutically acceptable excipients. More particularly, the present invention pertains to a vaccine composition comprising an immunoadjuvant compound as defined above, together with one or more antigens.

A variety of substances including cancer peptides and tumor associated antigens can be used as antigens in a compound or formulation, of immunogenic or vaccine type. An isolated antigen can be prepared using a variety of methods well known in the art. A gene encoding any immunogenic polypeptide can be isolated and cloned, for example, in bacterial, yeast, insect, reptile or mammalian cells using recombinant methods well known in the art and described, for example in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998). A number of genes encoding surface antigens from viral, bacterial and protozoan pathogens have been successfully cloned, expressed and used as antigens for vaccine development. For example, the major surface antigen of hepatitis B virus, HbsAg, the P subunit of choleratoxin, the enterotoxin of E. coli, the circumsporozoite protein of the malaria parasite, and a glycoprotein membrane, antigen from Epstein-Barr virus, as well as tumor cell antigens, have been expressed in various well known vector/host systems, purified and used in vaccines.

A pathologically aberrant cell may also be used in a vaccine composition according to the invention can be obtained from any source such as one or more individuals having a pathological condition or ex vivo or in vitro cultured cells obtained from one or more such individuals, including a specific individual to be treated with the resulting vaccine.

The vaccine composition according to the invention may contain at least one other immunoadjuvant. A variety of immunoadjuvant may be suitable to alter an immune response in an individual. The type of alteration desired will determine the type of immunoadjuvant selected to be combined with the said beta glucan compound of the invention. For example, to enhance the innate immune response, the vaccine composition of the invention can comprise another immunoadjuvant that promotes an innate immune response, such as other PAMP or conserved region known or suspected of inducing an innate immune response. A variety of PAMPs are known to stimulate the activities of different members of the toll-like family of receptors. Such PAMPs can be combined to stimulate a particular combination of toll-like receptors that induce a beneficial cytokine profile. For example, PAMPs can be combined to stimulate a cytokine profile that induces a Th1 or Th2 immune response. Other types of immunoadjuvant that promote humoral or cell-mediated immune responses can be combined with a cyclic beta glucan compound of the invention. For example, cytokines can be administered to alter the balance of Th1 and Th2 immune responses. Those skilled in the art will know how to determine the appropriate cytokines useful for obtaining a beneficial alteration in immune response for a particular pathological condition.

In another particular embodiment, the vaccine composition according to the invention, further comprises one or more components selected from the group consisting of surfactants, absorption promoters, water absorbing polymers, substances which inhibit enzymatic degradation, alcohols, organic solvents, oils, pH controlling agents, preservatives, osmotic pressure controlling agents, propellants, water and mixture thereof.

The vaccine composition according to the invention can further comprise a pharmaceutically acceptable carrier. The amount of the carrier will depend upon the amounts selected for the other ingredients, the desired concentration of the antigen, the selection of the administration route, oral or parenteral, etc. The carrier can be added to the vaccine at any convenient time. In the case of a lyophilized vaccine, the carrier can, for example, be added immediately prior to administration. Alternatively, the final product can be manufactured with the carrier. Examples of appropriate carriers include, but are not limited to, sterile water, saline, buffers, phosphate-buffered saline, buffered sodium chloride, vegetable oils, Minimum Essential Medium (MEM), MEM with HEPES buffer, etc.

Optionally, the vaccine composition of the invention may contain conventional, secondary adjuvants in varying amounts depending on the adjuvant and the desired result. The customary amount ranges from about 0.02% to about 20% by weight, depending upon the other ingredients and desired effect. Examples of suitable secondary adjuvants include, but are not limited to, stabilizers; emulsifiers; aluminum hydroxide; aluminum phosphate; pH adjusters such as sodium hydroxide, hydrochloric acid, etc.; surfactants such as Tween® 80 (polysorbate 80, commercially available from Sigma Chemical Co., St. Louis, Mo.); liposomes; iscom adjuvant; synthetic glycopeptides such as muramyl dipeptides; extenders such as dextran or dextran combinations, for example, with aluminum phosphate; carboxypolymethylene; bacterial cell walls such as mycobacterial cell wall extract; their derivatives such as Corynebacterium parvum; Propionibacterium acne; Mycobacterium bovis, for example, Bovine Calmette Guerin (BCG); vaccinia or animal poxvirus proteins; subviral particle adjuvants such as orbivirus; cholera toxin; N,N-dioctadecyl-N′,N′-bis(2-hydroxyethyl)-propanediamine (pyridine); monophosphoryl lipid A; dimethyldioctadecylammonium bromide (DDA, commercially available from Kodak, Rochester, N.Y.); synthetics and mixtures thereof. Desirably, aluminum hydroxide is admixed with other secondary adjuvants or 5 an immunoadjuvant such as Quil A.

Examples of suitable stabilizers include, but are not limited to, sucrose, gelatin, peptone, digested protein extracts such as NZ-Amine or NZ-Amine AS. Examples of emulsifiers include, but are not limited to, mineral oil, vegetable oil, peanut oil and other standard, metabolizable, nontoxic oils useful for injectables or intranasal vaccines compositions.

Conventional preservatives can be added to the vaccine composition in effective amounts ranging from about 0.0001% to about 0.1% by weight. Depending on the preservative employed in the formulation, amounts below or above this range may be useful. Typical preservatives include, for example, potassium sorbate, sodium metabisulfite, phenol, methyl paraben, propyl paraben, thimerosal, etc.

The vaccine composition of the invention can be formulated as a solution or suspension together with a pharmaceutically acceptable medium. Such a pharmaceutically acceptable medium can be, for example, water, phosphate buffered saline, normal saline or other physiologically buffered saline, or other solvent or vehicle such as glycol, glycerol, and oil such as olive oil or an injectable organic ester. A pharmaceutically acceptable medium can also contain liposomes or micelles, and can contain immunostimulating complexes prepared by mixing polypeptide or peptide antigens with detergent and a glycoside, such as Quil A.

Liquid dosage forms for oral administration of the vaccine composition of the invention include pharmaceutically-acceptable emulsions, microemulsions, solutions, suspensions, syrups and elixirs. In addition to the active ingredient(s), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and 5 mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Vaccine compositions of this invention suitable for parenteral administration comprise the active ingredient(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and non-aqueous carriers, which may be employed in the vaccine compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can 5 be maintained, for example, by the use of coating materials, such as lecithin, by the maintenance of the required particle size in the case of dispersions, and by the use of surfactants.

These compositions may also contain adjuvants such as wetting agents emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents, which delay absorption such as aluminum monostearate and gelatin. Injectable depot forms are made by forming microencapsule matrices of the active ingredient(s) in biodegradable polymers such as polylactidepolyglycolide. Depending on the ratio of the active ingredient(s) to polymer, and the nature of the particular polymer employed, the rate of release of the active ingredient(s) can be controlled. Examples of other biodegradable polymers include poly(orthoesters) and poly(anhydrides). Depot injectable formulations are also prepared by entrapping the active ingredient(s) in liposomes or microemulsions which are compatible with body tissue. The injectable materials can be sterilized for example, by filtration through a bacterial-retaining filter.

The formulations may be presented in unit-dose or multi-dose sealed containers, for example, ampoules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The amount of antigen and immunoadjuvant compound in the vaccine composition according to the invention are determined by techniques well known to those skilled in the pharmaceutical art, taking into consideration such factors as the particular antigen, the age, sex, weight, species, and condition of the particular animal or patient, and the route of administration.

While the dosage of the vaccine composition depends notably upon the antigen, species of the host vaccinated or to be vaccinated, etc., the dosage of a pharmacologically effective amount of the vaccine composition will usually range from about 0.01 μg to about 500 μg (and in particular 50 μg to about 500 μg) of the immunoadjuvant compound of the invention per dose.

Although the amount of the particular antigenic substance in the combination will influence the amount of the immunoadjuvant compound according to the invention, necessary to improve the immune response, it is contemplated that the practitioner can easily adjust the effective dosage amount of the immunoadjuvant compound through routine tests to meet the particular circumstances.

The vaccine composition according to the invention can be tested in a variety of preclinical toxicological and safety studies well known in the art. For example, such a vaccine composition can be evaluated in an animal model in which the antigen has been found to be immunogenic and that can be reproducibly immunized by the same route proposed for human clinical testing. For example, the vaccine composition according to the invention can be tested, for example, by an approach set forth by the Center for Biologics Evaluation and Research/Food and Drug Administration and National Institute of Allergy and Infectious Diseases (13).

Those skilled in the art will know how to determine for a particular vaccine composition, the appropriate antigen payload, route of immunization, volume of dose, purity of antigen, and vaccination regimen useful to treat a particular pathological condition in a particular animal species.

In a vaccination protocol, the vaccine may be advantageously administered as a unique dose or preferably, several times e.g., twice, three or four times at week or month intervals, according to a prime/boost mode. The appropriate dosage depends upon various parameters.

As a general rule, the vaccine composition of the present invention is conveniently administered orally, parenterally (subcutaneously, intramuscularly, intravenously, intradermally or intraperitoneally), intrabuccally, intranasally, or transdermally, intralymphatically, intratumorally, intravesically, intraperitoneally and intracerebrally. The route of administration contemplated by the present invention will depend upon the antigen.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,402,431: T-Cell Therapy Formulation.
U.S. Patent Application Publication No. 2011/0059054: Enhanced Dendritic Cells for Cancer Immunotherapy.

What is claimed is:

1. A therapeutic composition for a treatment of a tumor of epithelial origin in a human subject comprising one or more active agents that inhibit one or more cytokines that mediate a T helper 2 (Th2) inflammation in the tumor thereby transforming the Th2 inflammation into a tumor rejecting T helper 1 (Th1) type inflammation associated with the acquisition of CD8+ T cells able to reject cancer, wherein the active agent comprises natural or synthetic β-glucans; cyclic β-glucans; β-glucan derivatives; substituted β-glucans; or any combinations thereof optionally solubilized, dispersed, or suspended in a suitable medium in an amount sufficient to treat the tumor.

2. The composition of claim 1, wherein the substituted β-glucans comprise one or more substituents selected from the group consisting of succinyl (Suc) residues; phosphoglycerol residues (P-Gro); phosphoethanolamine (P-Etn) residues; phosphocholine residues (P-Cho); acetyl (Ace) residues; methylmalonyl (MeMal) residues; or any combinations thereof.

3. The composition of claim 1, wherein the β-glucans may be derived from one or more bacteria selected from the group consisting of Brucella abortus; Brucella melitensis, E. coli; Sinorhizobium melitoti; Bradyrhizobium japonicum; R. sphaeroides; or any combinations thereof.

4. The composition of claim 1, wherein the tumor is selected from a breast; prostate; lung;
colorectal; or pancreatic cancer.

5. The composition of claim 1, wherein the β-glucans are adapted to be administered intratumorally,

6. The composition of claim 1, further comprising at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

7. A method of treating or ameliorating symptoms of a cancer of epithelial origin in a human subject comprising the steps of:
identifying the subject in need of treatment against the cancer; and
administering a therapeutically effective amount of a pharmaceutical composition sufficient to treat or ameliorate the symptoms of the cancer in the subject comprising one or more natural or synthetic β-glucans; cyclic β-glucans; β-glucan derivatives; substituted β-glucans; or any combinations thereof optionally solubilized, dispersed, or suspended in a suitable medium.

8. The method of claim 7, wherein the substituted β-glucans comprise one or more substituents selected from the group consisting of succinyl (Suc) residues; phosphoglycerol residues (P-Gro); phosphoethanolamine (P-Etn) residues; phosphocholine residues (P-Cho); acetyl (Ace) residues; methylmalonyl (MeMal) residues; or any combinations thereof.

9. The method of claim 7, wherein the β-glucans may be derived from one or more bacteria selected from the group consisting of Brucella abortus; Brucella melitensis, E. coli; Sinorhizobium melitoti; Bradyrhizobium japonicum; R. sphaeroides; or any combinations thereof.

10. The method of claim 7, wherein the tumor is selected from a breast; prostate; lung; colorectal; or pancreatic cancer.

11. The method of claim 7, wherein the β-glucans are adapted to be administered intratumorally.

12. The method of claim 7, wherein the β-glucans are administered intratumorally.

13. The method of claim 7, further comprising the administration of at least one anti-cancer agent selected from the group consisting of chemotherapeutic anti-cancer agents, anti-cancer vaccines, target-specific anti-cancer agents, for separate, sequential, simultaneous, concurrent or chronologically staggered use in therapy.

14. An ex vivo cellular composition for providing an immunotherapy against one or more tumors of epithelial origin in a human subject comprising one or more β-glucan loaded dendritic cells (DCs) grown or cultured in a medium comprising one or more tumor antigens or factors.

15. The composition of claim 14, wherein the DCs comprise myeloid DCs (mDCs).

16. The composition of claim 14, wherein the composition inhibits one or more cytokines that mediate a T helper 2 (Th2) inflammation in the tumor thereby transforming the Th2 inflammation into a tumor rejecting T helper 1 (Th1) type inflammation.

17. The composition of claim 14, wherein the composition decreases a level of one or more cytokines mediating the Th2 inflammation, wherein the cytokines are selected from the group consisting of IL-4, TNFα, and IL-13 producing CD4+ T cells.

18. The composition of claim 14, wherein the composition increases a level or an expression of IL-12p70, CD103+ T cells, or both.

19. The composition of claim 14, wherein the tumor is selected from a breast; prostate; lung; colorectal; or pancreatic cancer.

20. A method for providing an adjuvant therapy in a human subject following or concurrently with a primary therapy against breast cancer comprising the steps of:
identifying the human subject suffering from breast cancer undergoing the primary therapy or who has undergone the primary therapy; and
administering a composition comprising one or more natural or synthetic β-glucans; cyclic β-glucans; β-glucan derivatives; substituted β-glucans; or any combinations thereof optionally solubilized, dispersed, or suspended in a suitable medium in an amount sufficient to reduce a tumor-induced inflammation, inhibit a tumor development, or both.

21. The method of claim 20, wherein the primary treatment comprises surgical intervention; chemotherapy; radiation therapy; immunotherapy; treatment with anti-cancer vaccines; target-specific anti-cancer agents; monoclonal antibodies; or any combinations thereof.

22. The method of claim 20, wherein the composition may be used to provide adjuvant therapy in one or more cancers selected from the group consisting of prostate; kidney; lung; bladder; colorectal; endometrial; melanoma; thyroid; brain; or pancreatic cancer.

23. The method of claim 20, wherein the β-glucans are adapted to be administered intratumorally.

24. The method claim 20, wherein the β-glucans are administered intratumorally.

25. A method of treating or ameliorating symptoms of a cancer of epithelial origin in a human subject comprising the steps of:
identifying the subject in need of treatment against the cancer; and
intratumorally administering a therapeutically effective amount of a pharmaceutical composition in an amount sufficient to treat or ameliorate the symptoms of the cancer in the subject comprising one or more natural or synthetic β-glucans; cyclic β-glucans; β-glucan derivatives; substituted β-glucans; or any combinations thereof optionally solubilized, dispersed, or suspended in a suitable medium adapted for intratumoral administration.

26. The method of claim 25, wherein the substituted β-glucans comprise one or more substituents selected from the group consisting of succinyl (Suc) residues; phosphoglycerol residues (P-Gro); phosphoethanolamine (P-Etn) residues; phosphocholine residues (P-Cho); acetyl (Ace) residues; methylmalonyl (MeMal) residues; or any combinations thereof.

27. The method of claim 25, wherein the β-glucans may be derived from one or more bacteria selected from the group consisting of Brucella abortus; Brucella melitensis, E. coli; Sinorhizobium melitoti; Bradyrhizobium japonicum; R. sphaeroides; or any combinations thereof.

28. The method of claim 25, wherein the tumor is selected from a breast; prostate; lung; colorectal; or pancreatic cancer.
